(19) United States
(12) Patent Application Publication
(10) Pub. No.: US 2005/0283074 A1
(43) Pub. Date: Dec. 22, 2005

(54) Ultrasound feedback for tissue ablation procedures

(75) Inventors: John I. Jackson, Menlo Park, CA (US); Bhaskar Ramamurthy, Los Altos, CA (US)

Correspondence Address: Attn: Elsa Keller;Legal Administrator, Intellectual Property Department, 170 Wood Avenue South, Iselin, NJ, 08830, US

(73) Assignee: Siemens Medical Solutions USA, Inc.

(21) Appl. No.: 10/875,010

(22) Filed: Jun. 22, 2004

(51) Int. Cl. A61B 8/00

(52) U.S. Cl. 600/439

(57) ABSTRACT

Bubble generation during a tissue ablation procedure is identified or detected. The ultrasound imaging is optimized to better detect generation of bubbles for more refined visualization and control of the ablation procedure. The generation of bubbles may alternatively or additionally be quantified to assist in control and/or diagnosis during an ablation procedure. Signals are generated based on the detection of a change in bubble characteristics. For example, the detection of type 2 or type 1 bubble generation is used to generate audio or visual warning signals. As another example, detection of type 1 or type 2 bubbles triggers generation of a control signal for increasing, decreasing or terminating the ablation energy. The generation of the control signal is performed automatically rather than relying on user visualization and reaction.

### BACKGROUND

The present invention relates to imaging in tissue ablation procedures. In particular, ultrasound imaging feedback is provided for tissue ablation procedures.

Radio frequency ablation of the pulmonary vein ostia is used to cure some cases of arterial fibrillation. The procedure is performed within an enclosed heart or chest by inserting an ablating catheter into a vein, such as the femoral vein, and passing the tip of the catheter into the right atrium of the heart through the interatrial septum, into the left atrium and up against the ostia of the pulmonary veins. The catheter tip is positioned using one or more of manual feel, fluoroscopy or ultrasound.

The ablation is performed by applying radio frequency energy to the tissue. The ablation is performed by controlling the power output of the ablation device. For example, 60 Watts may be applied for a period of 60 seconds. The clinical success of the procedure may be dependent on the physician's ability to deliver sufficient energy to the appropriate tissue to interrupt undesired electrical activity through the tissue. A possible complication or risk of the procedure is that too much application of energy may cause harm, possibly resulting in narrowing or stenosis of the pulmonary vein ostium.

Since the tissue undergoing the treatment cannot be directly visualized, other mechanisms are used for assessing the progress of the ablation procedure. For example, the electrical impedance between the ablation catheter and a grounding patch on the patient's back is measured. A sudden increase in the electrical impedance is typically indicative that tissue damage has begun. Unfortunately, at this point, undesired damage may have already occurred. To avoid undesired damage, a specific power and temporal procedure is predetermined. Generalized temporal or power settings may provide for incomplete or non-optimized ablation. Temperature limits may also be used, but the temperature may vary given the same power or energy depending on the patient and placement of the catheter. The catheter-to-tissue contact or placement of contact may also adversely affect the outcome of an ablation procedure.

N. Marrouche et al. in “Phase-Array Intra-Cardiac Echo Cardiographic Monitoring During Pulmonary Vein Isolation In Patients With Arterial Fibrillation,” 107 Circulation, pages 2710-2716, 2003, use ultrasound imaging to assist ablation procedures. Ultrasound imaging allows for real time monitoring of radio frequency energy delivery. Bubbles generated by the energy are viewed using fundamental B-mode imaging. Different types of bubbles are generated in different situations. FIG. 1 shows a graphical representation of the bubble signal level as a function of ablation energy. At moderate energy levels, type 1 or a sparse number of bubbles are generated. Approximately three to five seconds before undesired tissue damage occurs, type 2 bubbles are generated. Type 2 bubbles correspond to a brisk shower of dense microbubbles. Ablation energy is terminated when a user views type 2 bubbles in an ultrasound image. The ablation energy may be adjusted downwards when type 1 or type 2 bubble generation is observed by the user. Power adjustment guided by visualization of microbubble formation may reduce the risk of pulmonary vein stenosis and improves long term care.

### BRIEF SUMMARY

By way of introduction, the preferred embodiments described below include methods and systems for identifying or detecting bubble generation during a tissue ablation procedure. The ultrasound imaging is optimized to better detect generation of bubbles for more refined visualization and/or control of the ablation procedure. The generation of bubbles may alternatively or additionally be quantified to assist in control and diagnosis during an ablation procedure. In a further alternative or additional embodiment, signals are generated based on the detection of a change in bubble characteristics. For example, the detection of type 2 or type 1 bubble generation is used to generate audio or visual warning signals. As another example, detection of type 1 or type 2 bubbles triggers generation of a control signal for increasing, decreasing or terminating the ablation energy. The generation of the control signal is performed automatically rather than relying on user visualization and reaction.

In a first aspect, a method is provided for identifying bubble generation during a tissue ablation procedure. A fluid region adjacent to the tissue is imaged with ultrasound during application of ablation energy. Imaging is selectively enhanced for imaging bubbles during application of the ablation energy.

In a second aspect, a system is provided for identifying bubble generation during a tissue ablation procedure. The receive beamformer connects with the transducer. A detector connects with the receive beamformer. A control processor is operable in response to selection of an ablation procedure to cause the receive beamformer, the detectors or combination thereof to enhance imaging of bubbles during the ablation procedure.

In a third aspect, a method is provided for detecting bubble generation during a tissue ablation procedure. A fluid region adjacent to tissue is imaged with ultrasound during the tissue ablation procedure. A change in a bubble characteristic is detected with the processor.

In a fourth aspect, a system for detecting bubble generation during a tissue ablation procedure is provided. A receive beamformer connects with the transducer. A detector connects with the receive beamformer. A control processor is operable to detect generation of bubbles by ablation from data output by the detector, the receive beamformer, the transducer or combinations thereof. The control processor is operable to detect the generation of bubbles in response to selection of an ablation procedure.

The present invention is defined by the following claims, and nothing in this section should be taken as a limitation on those claims. Further aspects and advantages of the invention are discussed below in conjunction with the preferred embodiment and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a graphical representation of a relationship between bubble generation and ablation energy;

FIG. 2 is a block diagram of one embodiment of a system for identifying or detecting bubble generation during a tissue ablation procedure; and

FIG. 3 is a flow chart diagram of one embodiment of a method for identifying or detecting bubble generation during a tissue ablation procedure.

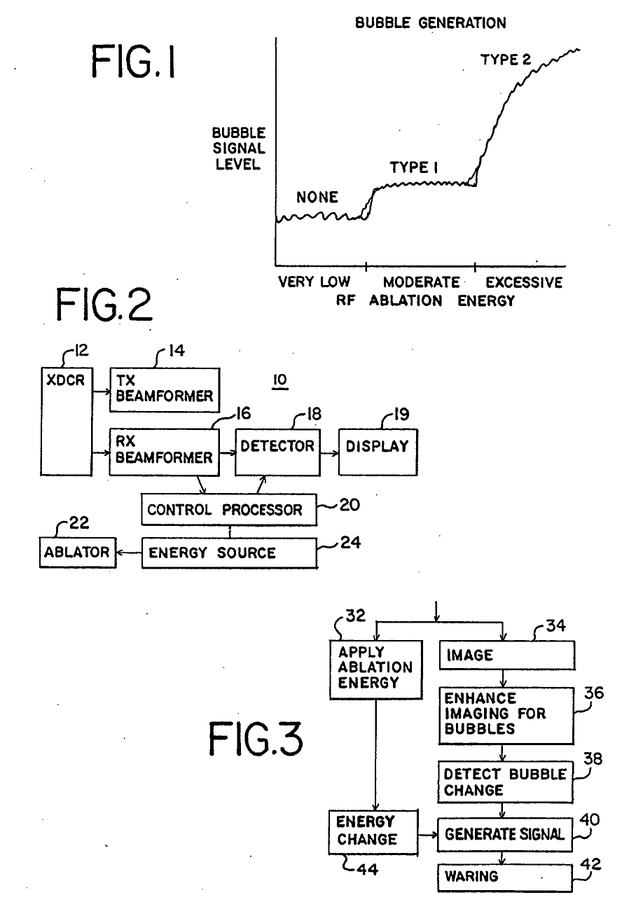

### DETAILED DESCRIPTION OF THE DRAWINGS AND PRESENTLY PREFERRED EMBODIMENTS

Improved ultrasonic monitoring of ablation procedures, such as intra-cardiac electrophysiology procedures, is provided. By analyzing an ultrasound image and controlling the source of power for the ablation device, a processor or other automated actions are provided. Feedback is used to control the energy deposition into the tissue. Ultrasonically bright reflectors, such as small gas bubbles, are generated during the ablation procedure. The rapid increase in the number of bright reflectors immediately precedes the onset of tissue damage. By monitoring the ultrasound image for different types of bubbles, the position or system may quickly reduce the ablation power before damage occurs. By increasing the specificity of the bubbles in ultrasound imaging, improved ablation procedure results may be provided. By automatically detecting an increase in the number of bubbles or other characteristic of bubbles, different actions to assist the ablation procedure may be performed. By alerting the user to the occurrence of a particular type of bubble or bubble characteristic, better control of ablation procedure may be provided. Direct electronic feedback may be used to control the power of the ablation device in response to detected characteristics of bubbles.

FIG. 2 shows one embodiment of a system 10 for identifying or detecting bubble generation during tissue ablation procedure. The system includes a transducer 12, a transmit beamformer 14, a receive beamformer 16, a detector 18, a display 19, a control processor 20, an ablator 22, and an energy source 24. Different, additional or fewer components may be provided, such as providing the system 10 without the ablator 22 and the energy source 24. The system 10 is a combined medical diagnostic ultrasound imaging system and an ablation catheter system. Alternatively, separate ablation and imaging systems are used.

For performing the ablation procedure, the ablator 22 is an ablation catheter for intra-cardiac use. Any now known or later developed ablation catheter may be provided. The energy source 24 is a source of radio frequency power, such as an alternating current source. Any of the frequency, power, peak-to-peak amplitude or other characteristics of the energy is controlled by the energy source 24. The energy source 24 provides ablation energy to the ablator 22. During use, the ablator 22 is positioned adjacent to the tissue, such as the pulmonary vein, for treatment of arterial fibrillation or other electrical malfunctions or disease states.

The transducer 12 is one or more piezoelectric or microelectro-mechanical elements for imaging, such as an intra-cardiac echocardiography imaging transducer. In one embodiment, the transducer 12 is a one-dimensional array of elements. For intra-cardiac use, the transducer 12 is an array of elements within an intra-cardiac catheter, such as the ACUSON AcuNav™ diagnostic ultrasound catheter from Siemens Medical Solutions, USA, Inc. The transducer elements are positioned near a tip of the catheter for imaging from within the cardiac system of a patient. In alternative embodiments, the transducer 12 is included in a transesophageal or a transthoraxic echo probe. In yet other embodiments, the transducer is within a probe housing shaped and sized for use externally to a patient. The transducer 12 of an intra-cardiac catheter is positioned adjacent to or within the same chamber or vascular structure as the ablator 22 for monitoring the ablation procedure. In one embodiment, the transducer 12 is operable to scan a volume rather than a planar region, such as a multi-dimensional array of elements or a one dimensional array that may be mechanically steered along elevation (e.g., a wobbler).

The transmit beamformer 14 generates one or more relatively delayed and apodized waveforms for imaging. The waveforms are applied to the transducer 12 for generating acoustic energy along one or more scan lines. By repeated application along different scan lines, a region of a patient is scanned with ultrasound energy. The ultrasound energy reflects off of tissue, fluids or other structures within a patient. Some of the reflections impinge on the transducer 12. The transducer 12 converts the received acoustic echoes into electrical signals. The electrical signals are provided to the receive beamformer 16.

The receive beamformer 16 is a processor, application specific integrated circuit, amplifier, filter, delay, summers, shift registers, multipliers, phase rotators, analog circuits, digital circuits, field programmable gate array, combinations thereof or other now known or later developed receive beamformer components. The receive beamformer 16 is configured into a plurality of channels. Each channel is associated with a different receive element in a receive aperture. The signals in each channel are relatively delayed and apodized. The relatively delayed and apodized are summed together to form a sample representative of a particular spatial location within the scan region. While shown directly connected with the transducer 12, the receive beamformer 16 may be indirectly connected with the transducer 12 through a transmit and receive switch or other components.

The receive beamformer 16 may include one or more filters positioned prior to or after the summer. The filters are operable to isolate signals at desired frequencies, such as harmonic frequencies. As used herein, harmonic frequencies include frequencies other than the transmitted fundamental frequency. For example, integer harmonics, ultra harmonics or sub-harmonics may be used. Finite impulse response, infinite impulse response or other filtering devices may be provided. In yet other embodiments, the receive beamformer 16 includes buffers, memory, multipliers, summers and/or filter devices for combining signals representing the same or adjacent spatial locations acquired at different times. For example, two or more sequential pulses are transmitted along a same or adjacent scan lines. The receive beamformed signals in response to each of the sequential pulses are then weighted or combined. By using relative phasing on the transmit beamformer 14, relative weighting in the receive beamformer 16, and/or relative phasing or positive or negative weighting in the receive beamformer 16, the desired information is enhanced or identified and undesired information may be reduced.

The detector 18 is a B-mode detector, Doppler detector, contrast agent detector, harmonic tissue detector, combinations thereof or another now known or later developed detector. In one embodiment, the detector 18 is an application specific integrated circuit, processor or other circuit for determining an intensity or energy associated with the receive signal. The detector 18 connects with the receive beamformer 16 directly or indirectly for obtaining the receive signals.

The detector 18 may include the filtering or other structure for combining multiple receive signals prior to detection described above for the receive beamformer 16. Alternatively or additionally, the detector 18 may include memories, buffers, multipliers, summers and/or filters for combining detected values associated with a same or similar spatial locations.

The control processor 20 is a general processor, digital signal processor, application specific integrated circuit, field programmable gate array, digital device, analog device, server, network, combinations thereof or other now known or later developed processor for controlling or interacting with one or more components of the system 10. The control processor 20 is responsive to selection of an ablation procedure. For example, connection of a specific transducer 12 to the system 10 triggers selection of an ablation procedure application by the control processor 20. As another example, connection of an energy source 24 or the ablator 22 where the energy source 24 is included in the imaging system causes the control processor 20 to implement selection of an ablation procedure application. As yet another example, a user configures the system 10 for operation with an ablation procedure, resulting in selection of the ablation procedure. Direct selection of an ablation procedure application may also be performed by the user.

In response to the selection of the ablation procedure, the control processor 20 causes the receive beamformer 16, the transmit beamformer 14, the detector 18 or combinations thereof to enhance imaging of bubbles during the ablation procedure. For example, the control processor 20 is operable to select an imaging process that enhances detectability of a bubble characteristic or other ablation indicator as opposed to fundamental B-mode imaging. For example, the receive beamformer 16 is configured to receive signals at a frequency different than a transmit frequency. Any of various harmonic receive frequencies may be implemented with the receive beamformer 16. Harmonic detection may be provided by the detector 18. The detector 18 and receive beamformer 16 may be configured to identify non-linear response to transmitted acoustic energy. The detector 18 and/or the receive beamformer 16 may provide weighted summation of the receive signals. A clutter filter of a Doppler detector 18 may be used to filter the receive signals to identify loss of correlation. The detector 18 may be configured for Doppler detection. Combinations of these various image enhancements to detect a bubble characteristic may be provided. Other now known or latter developed components and configurations of components may be used for enhancing imaging of bubbles during an ablation procedure. For example, contrast agent imaging techniques are performed despite a lack of user injected contrast agent.

The control processor 20 is additionally or alternatively operable to detect generation of bubbles or bubble characteristics caused by ablation from data output from the detector 18, the receive beamformer 16, the transducer 12 or combinations thereof. For example, the control processor 20 determines an increase in the number of bubbles as an increase in intensity of a region or entire image. A threshold increase in the number of bubbles may be used to trigger other actions. The control processor 20 may perform other quantifications to determine bubble characteristics.

In addition or as an alternative to configuring the system 10 for enhanced imaging of possible bubbles generated during the ablation procedure and/or detecting generation of bubbles or a bubble characteristic, the control processor 20 generates signals in response to detection of a threshold bubble characteristic or in response to bubbles generated during the ablation procedure. For example, the control processor 20 generates a visual warning signal display on the display 19 in response to a characteristic of the detected generation of the bubbles. An audible warning may be generated on a speaker in response to a characteristic of the detected generation of bubbles. For example, one type or level of warning is audio or visually provided based on the detection of type 1 bubbles or relatively sparse bubble generation. The user may then decrease an ablation power. A different type or higher level warning is provided in response to the detection of generation of type 2 bubbles or a large increase in the number of bubbles generated. The user or system 10 may then cease application of the ablation energy. As an alternative or in addition to generation of a warning signal, the control processor 20 outputs a control signal on a connection with the source 24 of ablation energy. The source 24 is responsive to the output signal to control the ablation energy, such as by increasing, decreasing or ceasing application of the energy to the ablator 22.

FIG. 3 shows one embodiment of a method for identifying or detecting bubble generation during a tissue ablation procedure. The method is implemented using the system 10 described above with respect to FIG. 2 or a different system. Additional, different or fewer acts may be provided in the same or different order than shown in FIG. 3. For example, only one, only two or all three of acts 36, 38 and 40 are provided. Acts 42 and 44 correspond generally to act 40 so may not be used when act 40 is not provided.

In act 32, ablation energy is applied. The ablation energy is applied to tissue. For example, a radio frequency energy sufficient to provide around 60 watts or other amounts of energy is applied to tissue. An ablation catheter or other electrode is positioned next to the desired tissue. A grounding patch is positioned on the patient or the patient is otherwise connected with a ground potential. The energy provided in the ablation catheter or electrode transfers through the tissue to ground. Any now known or later developed technique for applying ablation energy may be used.

In act 34, a fluid region adjacent to tissue is imaged with ultrasound during application of the ablation energy. For example, intra-cardiac echocardiography is provided from an intra-cardiac catheter. Transducers within the catheter generate acoustic energy and receive acoustic echoes for forming an image. One-dimensional, two-dimensional or three-dimensional imaging may be used. By using three-dimensional imaging, the imaged region more likely includes fluid regions where bubbles may be generated during ablation. By positioning a catheter or transducer adjacent to the tissue to be treated, the imaging plane or other region is positioned to include both the tissue as well as fluid adjacent to the tissue where bubble generation is most likely to occur. For B-mode imaging, the tissue appears as a generally bright to mid-range reflectors while fluid appears to be dark to mid-range reflectors. Other types of imaging, such as Doppler imaging with greater signals for fluid than for tissue, may be used. The fluid and tissue regions are repetitively scanned during the tissue ablation procedure. A sequence of images is provided during the procedure.

In act 36, the imaging of act 34 is selectively enhanced for imaging bubbles during the tissue ablation procedure. The imaging is adapted to detect bubbles. For example, the conspicuity of any bubbles is increased by selection of a receive signal process, such as a process associated with contrast agent imaging. The enhancement makes any bubbles or microspheres more likely to stand out or have contrast with tissue or blood as compared to fundamental B-mode imaging. Any of various imaging processes may be used alone or in combination. For example, signals received at frequencies other than the transmitted frequency are used for imaging. Signals at any harmonic, ultra harmonic, or sub-harmonic using either single pulse filtering or multiple pulse (e.g., pulse inversion or phase inversion) combination may be used. Signals are received at a harmonic frequency band of the transmit frequency band, such as receiving signals in a frequency band around the second harmonic of the transmitted frequency band. Weighted summation of receive beamformed signals or other return signals may be used, such as disclosed in U.S. Pat. No. 6,494,841, the disclosure of which is incorporated herein by reference. The techniques disclosed in U.S. Pat. Nos. 6,436,041 and 6,497,666, the disclosures of which are incorporated herein by reference, may additionally or alternatively be used. Echo signals from a plurality of transmissions are combined for each of a plurality of spatial locations. The non-linear response of the fluid or tissues is detected in response to the weighted summation or combination of information. The non-linear response is at the transmitted fundamental frequency or at another frequency. As another imaging process, Doppler detection is used. The loss of correlation of bubbles due to destruction and/or the general speed difference between the movement of bubbles versus fluid and tissue may be used to enhance imaging of the bubbles through Doppler energy or velocity detection. A clutter filter, such as used in Doppler imaging, may be used to suppress strong signals from the relatively slow moving myocardium and other tissue while allowing signals from bubbles to remain. The clutter filter is used for B-mode imaging or Doppler imaging to enhance the imaging for bubbles.

Any now known or later developed contrast agent imaging technique may be used to enhance the detection of any bubbles during an ablation procedure without the user injection of additional contrast agents. For example, any of Phase Inversion, Pulse Inversion, Power Pulse Inversion, Ensemble Contrast Imaging, Power Harmonics, Power Angio, Power Modulation, Ultra Harmonics, Flash Echo Imaging, Advanced Dynamic Flow, 1.5 Harmonic Imaging, Coherent Contrast Imaging, Contrast Pulse Sequencing, Power Contrast Imaging, and Agent Detection Imaging may be used. The terms above are used by various ultrasound equipment manufacturers for contrast agent imaging techniques. Any of various high power, low power, types of transmitted pulses, the number of transmitted pulses per line in an image, the type of filter, receive frequency content, the type of filtering across all receive pulses, the type of detection, the type of weighting, the relative phasing between transmitted pulses, the relative polarity between transmitted pulses, the polarity of weights in received processing, the relative weighting in received processing, combinations thereof or other characteristics are varied to enhance the imaging of bubbles. Any of these techniques may be used alone or in combination. Additional techniques disclosed in U.S. Pat. No. ______ (application Ser. No. 10/644,862), the disclosure of which is incorporated herein by reference, may be used.

Different regions of images may be enhanced for different purposes. For example, a region of interest associated with the fluid or tissue is identified. Within the region of interest, a desired type of imaging is used. For example, image processing to enhance images of bubbles is provided for a fluid region regardless of the existence of bubbles in the region and separate or different image processing is provided for tissue regions. In yet another embodiment, the entire scan region is imaged with or without image processing adapted for enhancing the contrast of bubbles. Once bubbles are detected, an overlay is provided on the image for enhancing the contrast or otherwise identifying the newly detected bubbles. For example, a black and white B-mode image is displayed. Once bubbles are detected, the pixels associated with the detected bubbles are highlighted using a color or increased brightness as an overlay or modulation on the B-mode image. The detection of the bubbles is performed using the enhanced image processing for imaging bubbles.

In act 38, a change in a bubble characteristic is detected with a processor. Signal or image processing is used to automatically detect the change. The change detected may be going from no bubbles to some bubbles, an increase in the number of bubbles, a difference in location of bubbles, a difference in size of bubbles, a difference in density of bubbles, or other characteristic of a single or group of bubbles. Using a processor, such as the control processor 20, to detect the change in bubble characteristic may allow for more immediate reaction, automatic signal generation, reliable quantification or reproducibility.

In one embodiment, the region of interest, such as a fluid region, is automatically determined based on variation of signals for different spatial locations as a function of time. As a result, the region of interest is automatically positioned within each image in a sequence of images so that patient movement or transducer movement is less likely to result in incorrect indication of bubbles. For each pixel on a B-mode or other image, the peak-to-peak, maximum, average or other variation is determined. In one embodiment, the variation is determined of a function of a cycle, such as a cardiac cycle. The variation is determined over one or other integral number of cardiac cycles. In another embodiment, a best fit sinusoid is used to determine the variation in brightness for each pixel as a function of the cardiac cycle. Pixels or spatial locations that consistently represent blood pool have little or no variation. Pixels from tissue and pixels that some time show tissue and sometimes show blood may have a greater amount of variation. The region of interest is set at spatial locations associated with the center in depth and laterally within the scanned region. Within that range, pixels or spatial locations associated with consistent representation of blood pool are identified as the region of interest. Preset or user defined angles or depth of an image may be used alternatively or additionally. A bubble characteristic is then measured using signal processing within the region of interest.

The change in characteristics of bubbles is detected using any characteristic of the receive signals. For example, an intensity characteristic is calculated for at least the fluid region. An increase in signal intensity within the fluid region, a predefined region, a user defined region of interest, the tissue region or elsewhere indicates the existence or magnitude of type 1 or type 2 bubbles. Any of various measures may be used, such as an integrated sum, a mean, a medium, a number of pixels or spatial regions above a predefined or user defined threshold or other statistic. For example, one or more of the region of interest measurements disclosed in U.S. Pat. No. 6,030,344, the disclosure of which is incorporated herein by reference, is used. The measurement of the characteristic may be associated with a single image or may be filtered over multiple images to reduce the effect of random noise. Filtering may vary as a function of the portion of the cardiac cycle associated with acquisition of a particular image to account for normal brightness variations within the cardiac cycle. Any of various temporal or spatial filtering may be used, such as low-pass temporal filtering.

The intensity characteristic or other measure is compared to a threshold. The threshold is predefined, application specific, patient specific or user set. The threshold may be adaptive as a function of received data. The threshold determines the levels or bubble characteristics associated with particular events. For example, one threshold is associated with detection of type 1 bubbles. Different thresholds may be provided for different levels of type 1 bubbles. Type 1 bubbles are bubbles associated with relatively sparse or minimal occurrence, such as bubbles generated for high ablation power close to but not causing tissue damage. An additional threshold or group of thresholds may be used for type 2 bubbles, or bubbles associated with a greater risk of tissue damage. Type 2 bubbles are denser within the fluid region near the ablation. Different lengths, actions, image processing or other outputs result from the comparison of the different measurements to thresholds. The user can set a nominal signal base line level to indicate normal, or sub-critical, occurrence of bubbles, a range above a base line number of bubbles, critical bubble level or other occurrence. Feedback to the user, such as a graph, number or color indicating a current signal level associated with bubbles and how the signal level compares with a base line and critical thresholds may be provided.

Other measurements and associated thresholds may be used. For example a topological analysis of an image is used to determine an increase in, decrease in, or the number of bright peaks within a spatially filtered or smooth image. For example, the number of signal peaks within the fluid region is determined. A larger number of peaks more likely indicate a greater number of bubbles or greater density of bubbles, indicating excessive ablation energy. Spatial filtering, such as with a low pass filter, avoids noise based peaks.

In act 40, an electric signal is generated in response to a bubble characteristic. For example, an electrical signal is generated in response to detected change in a bubble characteristic or in response to comparison of a bubble characteristic to a threshold. An electrical signal corresponds to a signal for providing feedback to a user or medical professional. Warnings are provided in act 42. For example, a visual warning is generated. An increase to type 2 bubble characteristics or number of bubbles may provide from two to five seconds of time until tissue damage occurs. A visual warning makes it more likely that a user can reduce or eliminate the ablation energy, avoiding tissue damage. The visual warning may be an icon, a flashing screen, a message on the screen, changing the color of an image, or other visual warning. For example, the detected bubbles are increased in brightness or otherwise highlighted to more effectively show the warnings. Alternatively or additionally, an electrical signal is generated as an audio warning in response to the threshold increase in bubble characteristic. A verbal warning, a warning sound or other audio warning is provided to the user. Warnings associated with other occurrences than a critical increase in bubbles may be provided. For example, warnings associated with reducing ablation energy in response to the onset of sparsely spaced or type 1 bubbles are provided.

In act 44, the signal generated in act 40 is alternatively or additionally output to change the ablation energy. For example, a signal indicating a desired change in the ablation energy is output from an ultrasound imaging system. The electrical signal is operable to cause the source of ablation energy to alter the output energy. A signal is used to turn up, turn down or turn off the ablation energy. Other alterations of the ablation energy, such as the frequency, amplitude, power or duration of application of the ablation energy may be changed. In one embodiment, the detection of sparsely spaced bubbles is used to indicate a decrease in power. A detection of type 2 or distribution of bubbles causes the source to cease application of any ablation energy. As other examples, the electrical signal may cause an increase in the ablation energy in response to a detected decrease in bubble signal level or detection of no bubble signal. Predefined or user set limits or adjustments are used for the amount of alteration. The generation of the signal in act 40 provides a safety net or automated mechanism responsive to an ultrasound image which may reduce adverse effects.

In one embodiment, the electrical signal generated to control the source of ablation energy or an electrical signal that is the source of ablation energy is provided using a transducer interface, such as an interface on an ultrasound system used for powering a transesophageal transducer, a wobbler transducer, or other transducer that includes a motor. An external mixing box or signal converter may additionally be used for converting any electrically output signal to a signal recognized by the ablation catheter or source of ablation energy.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for identifying bubble generation during a tissue ablation procedure, the method comprising:
(a) imaging a fluid region adjacent to the tissue with ultrasound during the tissue ablation procedure; and
(b) selectively enhancing the imaging for imaging bubbles during (a).

2. The method of claim 1 wherein (a) comprises performing intra-cardiac echography while radio frequency energy is applied to tissue.

3. The method of claim 1 wherein (a) comprises imaging both the tissue and the fluid region adjacent to the tissue.

4. The method of claim 1 wherein (a) comprises imaging from an intra-cardiac catheter.

5. The method of claim 1 wherein (b) comprises increasing conspicuity of any bubbles by selection of a received signal process.

6. The method of claim 1 wherein (b) comprises selecting an imaging process from the group of: receiving signals at a frequency different than the transmit frequency, harmonic imaging, detecting non-linear response to transmitted acoustic energy, weighted summation of receive beamformed signals, clutter filtering received signals, Doppler detection and combinations thereof.

7. The method of claim 1 wherein (b) comprises receiving signals at a harmonic frequency band of a transmit frequency band.

8. The method of claim 1 wherein (b) comprises combining echo signals from a plurality of transmissions for each of a plurality of spatial locations.

9. The method of claim 8 wherein (b) comprises detecting a non-linear response.

10. The method of claim 1 wherein (b) comprises suppressing at least some signals from tissue.

11. The method of claim 10 wherein (b) comprises clutter filtering received signals.

12. The method of claim 1 further comprising:
(c) detecting an increase in a number of bubbles with a processor.

13. The method of claim 1 further comprising:
(c) generating an electric signal in response to a bubble characteristic.

14. A system for identifying bubble generation during a tissue ablation procedure, the system comprising:
a transducer;
a receive beamformer connected with the transducer;
a detector connected with the receive beamformer; and
a control processor operable in response to selection of an ablation procedure to cause the receive beamformer, the detector or combinations thereof to enhance imaging of bubbles during the ablation procedure.

15. The system of claim 14 wherein the transducer comprises a transducer in an intra-cardiac echography catheter;
further comprising:
a tissue ablation catheter adjacent to the intra-cardiac catheter.

16. The system of claim 14 wherein the control processor is operable to select an imaging process from the group of: receiving signals at a frequency different than the transmit frequency with the receive beamformer, harmonic imaging with the receive beamformer, harmonic detection with the detector, detecting non-linear response to transmitted acoustic energy with the detector, weighted summation of receive beamformed signals with the detector, clutter filtering received signals with the detector, Doppler detection with the detector and combinations thereof.

17. The system of claim 14 wherein the control processor is operable to determine a threshold increase in a number of bubbles.

18. The system of claim 14 further comprising:
a control signal output connected with the control processor and a source of radio frequency ablation energy, the source responsive to a signal on the control signal output.

19. A method for detecting bubble generation during a tissue ablation procedure, the method comprising:
(a) imaging a fluid region adjacent to the tissue with ultrasound during the tissue ablation procedure; and
(b) detecting a change in a bubble characteristic with a processor.

20. The method of claim 19 wherein (a) comprises performing intra-cardiac echography while radio frequency energy is applied to tissue.

21. The method of claim 19 wherein (a) comprises imaging from an intra-cardiac catheter.

22. The method of claim 19 wherein (b) comprises detecting an increase in a number of bubbles.

23. The method of claim 19 wherein (b) comprises calculating an intensity characteristic for at least the fluid region.

24. The method of claim 23 wherein (b) comprises comparing the intensity characteristic to a threshold.

25. The method of claim 19 wherein (b) comprises determining a number of signal peaks in at least the fluid region.

26. The method of claim 19 wherein (b) comprises determining variation for a plurality of spatial locations as a function of time.

27. The method of claim 19 further comprising:
(c) generating an electric signal in response to the detected change.

28. The method of claim 27 wherein (c) comprises generating a visual warning in response to a threshold increase in the bubble characteristic.

29. The method of claim 27 wherein (c) comprises generating an audio warning in response to a threshold increase in the bubble characteristic.

30. The method of claim 27 wherein (c) comprises outputting the electrical signal to a source of the ablation energy, the electrical signal operable to cause the source to alter the ablation energy.

31. The method of claim 30 wherein the electrical signal is operable to cause the source to cease application of the ablation energy.

32. The method of claim 19 further comprising:
(c) adapting imaging to detect bubbles during the tissue ablation procedure.

33. A system for detecting bubble generation during a tissue ablation procedure, the system comprising:
a transducer;
a receive beamformer connected with the transducer;
a detector connected with the receive beamformer; and
a control processor operable to detect generation of bubbles by ablation from data output by the detector, the receive beamformer, the transducer or combinations thereof, the control processor operable to detect the generation of bubbles in response to selection of an ablation procedure.

34. The system of claim 33 wherein the transducer comprises a transducer in an intra-cardiac echography catheter;
further comprising:
a tissue ablation catheter adjacent to the intra-cardiac catheter.

35. The system of claim 33 wherein the control processor is operable to select an imaging process from the group of: receiving signals at a frequency different than the transmit frequency with the receive beamformer, harmonic imaging with the receive beamformer, harmonic detection with the detector, detecting non-linear response to transmitted acoustic energy with the detector, weighted summation of receive beamformed signals with the detector, clutter filtering received signals with the detector, Doppler detection with the detector and combinations thereof.

36. The system of claim 33 wherein the control processor is operable to determine a threshold increase in a number of bubbles.

37. The system of claim 33 further comprising:
a control signal output connected with the control processor and a source of radio frequency ablation energy, the source responsive to a signal on the control signal output
wherein the control processor is operable to generate the signal in response to detection of a threshold bubble characteristic.

38. The system of claim 33 further comprising:
a display operable to display a warning in response to a characteristic of the detected generation of the bubbles.

39. The system of claim 33 further comprising:
a speaker operable to audibly warn in response to a characteristic of the detected generation of the bubbles.

40. A method for detecting bubble generation during a tissue ablation procedure, the method comprising:
(a) imaging a fluid region adjacent to the tissue with ultrasound during the tissue ablation procedure; and
(b) automatically generating at least one of a warning signal, an ablation energy control signal or combinations thereof in response to processor identified bubbles in the fluid region.

41. The method of claim 1 wherein (a) comprises three-dimensional imaging of the fluid region.
